(19) United States
(12) Patent Application Publication
(10) Pub. No.: US 2023/0116004 A1
(43) Pub. Date: Apr. 13, 2023

(54) SYSTEMS AND METHODS FOR PHYSICAL THERAPY USING AUGMENTED REALITY AND TREATMENT DATA COLLECTION AND ANALYSIS

(71) Applicant: SELFIT MEDICAL LTD., Ramat Hasharon (IL)

(72) Inventors: Almog NEUBERGER, Tel Aviv (IL); Avi Mordechai SHARIR, Ramat Hasharon (IL); Shahar FIGELMAN, Tel Aviv (IL); Amit KATZ, Gedera (IL)

(21) Appl. No.: 18/080,106

(22) Filed: Dec. 13, 2022

Related U.S. Application Data
(63) Continuation-in-part of application No. 16/481,868, filed on Jul. 30, 2019.

(30) Foreign Application Priority Data
Mar. 22, 2017 (IL) 251340

(51) Int. Cl. A63B 71/06; A63B 24/00

(52) U.S. Cl. CPC A63B 71/0622; A63B 24/0075; A63B 24/0062; A63B 2230/605; A63B 2220/807; A63B 2230/625

(57) ABSTRACT

The present invention relates to systems and methods for physical therapy in augmented reality (AR) comprising: at least one projector; at least one motion capture sensor; at least one surface EMG sensor, balance sensing unit; a computer in connection to said at least one projector, at least one motion capture sensor, and at least one surface EMG sensor and balance sensing unit. Wherein, movement and actions instructions are provided to a patient in visual and cues projected on a surface in patient's vicinity; and further wherein, data regarding patient's movements and actions are collected by said at least one motion capture sensor and at least one surface EMG sensor and balance sensing unit. It is within the provision of the invention that said data is processed and analyzed into a representation of patient's status and progress, further providing suitable therapy and exercise plans.

### RELATED APPLICATIONS

This application is filed as a Continuation-in-Part of US Patent Application No. U.S. Ser. No. 16/481,868, filed on Jul. 30, 2019, which is a National Phase of PCT Patent Application No. PCT/IL2018/050136 having International filing date of Feb. 7, 2018, which claims the benefit of priority of Israeli Application No. 251340 filed on Mar. 22, 2017. The contents of the above applications are incorporated by reference as is fully set forth in their entirety.

### FIELD OF THE INVENTION

The present invention relates generally to the field systems and methods for physical therapy and rehabilitation. More specifically, the present invention relates to the field of physical therapy and rehabilitation systems and methods involving augmented reality and treatment feedback data collection and analysis.

### BACKGROUND OF THE INVENTION

Physical therapy aims to remedy physical impairment in patients and promotes better mobility and function through examination, movement exercise, and application of force.

Professionals in the field deal daily with various shortcomings that prevent them from maintaining a structured treatment plan, evaluation of patient status and progress, which may ultimately result in patient rehabilitation failure.

Traditionally, physiotherapists rely on their experience and have limited or no means to empirically evaluate patient's status and progress. This affects the professionals' ability to make consistent and accurate therapy plans and may ultimately result in waste of time and effort of both the patient and the therapist.

Additionally, physical therapy is inherently uncomfortable experience as it forces the patient outside of his comfort zone. This problem results in motivation problems of the patient which may ultimately cause the patient skip therapy sessions and reduce adherence of the rehabilitation process.

Several attempts have been made to address the above problems. One such attempt involves a virtual reality (VR) system that is comprised of a computer, a screen for displaying interactive and engaging content and a markerless motion capture sensor comparable to Microsoft Kinect™. A patient follows movement instructions displayed on the screen, while the system records and analyzes patient's movements. This way, the system follows patient's status and progress and allows the therapist some degree of control over patient rehabilitation process.

Yet, several shortcomings and deficiencies in the field still remain, for example:
a. Existing attempts collect mostly visual data regarding patient's movement. This type of data does not allow identifying and tracing the origin of the impairment in cases where nerve damage is involved (e.g. strokes). Analysis of only visual data does not allow verifying if a patient indeed tries to activate the correct muscles or tries to avoid activating the muscle because of pain or inability.
b. Existing attempts engage with patients via cues displayed on a screen. This prevents patients from engaging with the real physical world (i.e., floor, table) rather with virtual cue received on a displayed screen. Interacting with the real physical world is a seamless experience which reflects real world environment and challenges, therefore places a lower barrier for the patient to adhere with the rehabilitation process as well as incentivized the patient to persist.
c. Additionally, wearable devices that need to be placed on the body of the patient are uncomfortable to use especially when there is some disability and physical inconvenience.

In light of the above description of the current state of the art, it is clear that there is a long-standing need for a solution that employs a different approach to resolve the issues and deficiencies in existing attempted solutions in the field.

### SUMMARY OF THE INVENTION

The present invention relates to computerized devices for physical therapy in augmented reality (AR) comprising:
at least one camera sensor and at least one projector for projecting visual cues on surfaces in a patient's vicinity;
wherein, processing circuitry of said computerized device is configured to:
define physical characteristics of said patient based on data captured with said at least one camera sensor;
dynamically generate a map of said vicinity of said patient including floor area and object properties data;
select and adjust a therapy exercise for a physical therapy session based on: the physical characteristics of said patient, clinical information of said patient, the dynamic map of the vicinity of said patient, and dependency conditions of said therapy exercise that are selected from the group comprising: minimum floor area, maximum floor area, properties of objects in the area, surfaces colors and textures in the area, and lighting in the vicinity;
instruct said at least one projector to project visual cues of the selected therapy exercise as movement and action instructions on said surfaces in said patient's vicinity.

It is within the provision of the invention that the computerized device comprising instructions that cause the processing circuitry of said computerized device to:
collect data regarding said patient's movements and action data in relation to said visual cues from said at least one camera sensor, an at least one surface EMG sensor, and a balance sensing unit, and store the collected data in a database;
compare said collected data with data of movements and action data collected during previous sessions of said patient;
instruct said at least one projector to project visual cues on said surfaces in said patient's vicinity as feedback regarding said patient's performance in relation to said movement and action instruction; and
based on comparison of said collected data to previously collected data, adjust said patient's therapy plan, movement and action instructions.

It is within the provision of the invention that said data is processed and analyzed into a representation of patient's status and progress, and further cross referenced with a data-base of representations of other patients' status and progress, further providing suitable therapy and exercise plans.

A method for physical therapy in augmented reality, comprising steps of:
defining physical characteristics of a patient based on data captured with at least one camera sensor;
dynamically generating a map of a vicinity of said patient including floor area and object properties data;
selecting and adjust a therapy exercise for a physical therapy session based on: the physical characteristics of said patient, clinical information of said patient, the dynamic map of the vicinity of said patient, and dependency conditions of said therapy exercise that are selected from the group comprising: minimum floor area, maximum floor area, properties of objects in the area, surfaces colors and textures in the area, and lighting in the vicinity;
providing movement and action instructions according to the selected therapy exercise by projecting visual cues on adjacent surfaces to said patient using at least one projector;
collecting data regarding the patient's movements and action data from at least one camera sensor, at least one balance sensing unit, and at least one surface EMG sensor;
evaluating said patient's movements and action data in relation to said movement and action instructions;
providing feedback regarding said patient's movement and actions by projecting visual feedback on said adjacent surfaces; and
adjusting said patient's therapy and exercise plans.

### BRIEF DESCRIPTION OF THE DRAWINGS

Embodiments and features of the present invention are described herein in conjunction with the following drawings:

FIG. 1 is a diagram depicting a system of the present invention.

FIG. 2 is a flowchart of a method of the present invention.

FIG. 3 is a flowchart of a method of the present invention.

FIG. 4 is a flowchart of a method of the present invention.

FIG. 5 is a diagram depicting an embodiment of a device of the present invention.

FIG. 6 is a flowchart of a method of the present invention.

FIG. 7 is a flowchart of a method of the present invention.

FIG. 8 is a diagram depicting a therapeutic exercise executed by an embodiment of the present invention.

FIG. 9 is a diagram depicting a therapeutic exercise executed by an embodiment of the present invention.

FIG. 10 is a diagram depicting an embodiment of a device of the present invention.

FIG. 11 is a diagram depicting an embodiment of a device of the present invention.

FIG. 12 is a diagram depicting an embodiment of a device of the present invention.

FIG. 13 is a diagram depicting an embodiment of a device of the present invention.

FIG. 14 is a diagram depicting an embodiment of a device of the present invention.

FIG. 15 is a diagram depicting a therapeutic exercise executed by an embodiment of the present invention.

FIG. 16 is a diagram depicting a therapeutic exercise executed by an embodiment of the present invention.

FIG. 17 is a diagram depicting a therapeutic exercise executed by an embodiment of the present invention.

FIG. 18 is a diagram depicting a therapeutic exercise executed by an embodiment of the present invention.

FIG. 19 is a diagram depicting a therapeutic exercise executed by an embodiment of the present invention.

FIG. 20 is a diagram depicting a therapeutic exercise executed by an embodiment of the present invention.

### DETAILED DESCRIPTION OF PREFERRED EMBODIMENTS

The present invention will be understood from the following detailed description of preferred embodiments, which are meant to be descriptive and not limiting. For the sake of brevity, some well-known features, methods, systems, procedures, components, circuits, and so on, are not described in detail.

The present invention provides many advantages over the prior art, among others for example, the following:
a. Systems of the present invention may select therapy exercises based on a dynamically generated map of a room where the therapy session is planned including the existence of various objects (e.g., balls, cups, boxes, and etc.) and their physical properties such as for example shape, dimensions, color and texture. This feature of the present invention may allow for a portable system that may be placed in many various locations including a patient's living room without any effort to setup and place cameras and projectors.
b. The system collects not only visual data regarding patient's movement and activity but may also collect data for example regarding the electrical activity of the patient's muscles and patient's body balance. Analysis of this combined data feed may allow identifying and tracing the origin of the impairment in cases where nerve damage is involved (e.g. strokes) or where the deficiency of patient's body balance is caused by asymmetry in the patient's body. This approach also may allow a more accurate assessment and scalable treatment of common impairments in a cost-effective manner.
c. Furthermore, analysis of a combination of visual with electrical muscle activity data may allow verifying if a patient indeed tries to activate the correct muscles or tries to avoid the muscle because of pain or inability.
   For example, if a patient walks without correctly shifting his weight between strides, a system of the present invention may be capable to identify this deficiency even if the patient succeeds in reaching his designated goals.
d. The present invention does not engage patients through a two-dimensional screen where experience is limited and not seamless, but rather through a projector that is capable of projecting visual content on all surfaces in patient's vicinity. This opens new and unique uses and possibilities.
   For example, projecting foot print images on a floor instructing patients to perform specific strides at a chosen pace.
   Another example involves projecting the patient's image in a specific position on a wall, encouraging the patient to adapt to the position while providing a visual indication on the same wall regarding the patient's position in relation to the provided instructions.

Another example involves projecting a virtual object on a table, encouraging the patient to move a physical object (e.g. a cup) from one place to another.

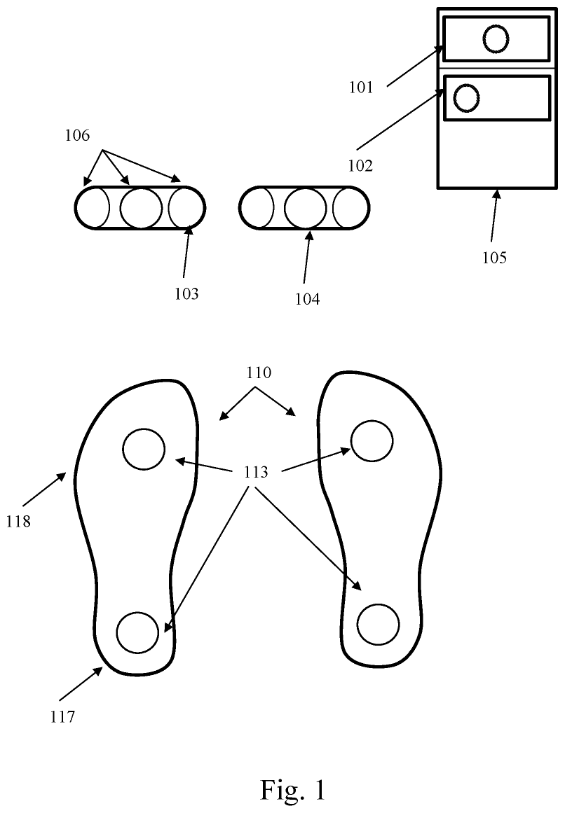

FIG. 1 depicts a system for physical therapy in augmented reality (AR) comprising:
a. one projector 101 that is capable of projecting visual cues on top of adjacent surfaces at normal interior lighting conditions.
b. one camera sensor 102; for example, a digital camera component such as Microsoft Kinect™ sensor having 3d image capture capabilities is suitable for this purpose but it should be mentioned that other types of 2D cameras and 3D modelling capture components such as Lidar sensors may be suitable to fill this purpose.
c. For example, two surface EMG sensors 103, 104; This embodiment uses a limb band with a MyoWare™ EMG sensor coupled with several surface electrode pads 106 dispersed in the perimeter of said limb band.
   The surface EMG sensor may optionally further comprise a vibrating component (not shown) for providing feedback to a patient. Considering a patient wearing an EMG sensor with a vibrating component on each limb. If the system would determine that the patient is not properly activating a muscle in a certain limb, the system shall activate the vibrating component that is attached to the limb with the deficiency.
   The surface EMG sensor may optionally further LED lights (not shown) for providing feedback to a patient. Considering a patient wearing an EMG sensor with LED lights on each limb. If the system would determine that the patient is not properly activating a muscle in a certain limb, the system shall activate the LED lights that are attached to the limb with the deficiency.
d. a balance sensing unit 110;
   In this embodiment, the balance sensing unit is comprised of four pressure sensors 113 distributed evenly in 2 insoles for insertion in a patient's shoes.
   FlexiForce™ pressure sensors may be suitable for this purpose but it should be mentioned that pressure sensors of other types and makes may be also suitable to fill this purpose. It should be mentioned that Tekscan iShoe™ insoles may be adapted for use in the system of the present invention without undue experimentation on-behalf of a person of average skill in the field of the present invention.
   One such pressure sensor may be built into the heel of the insole 117 while the other pressure sensor may be built into the fore of the insole 118. Each insole may further comprise a battery, a microprocessor, a memory unit, and wireless communication module for transmitting collected data to the system (not shown).
   This configuration may allow the inventive system to gather data regarding patient's weight distribution in movement.
e. A computerized device 105 in connection to said projector, camera sensor, two surface EMG sensors, and balance sensing unit.
   The computerized device may be integrated with the projector and camera sensor in a single device and wirelessly connected with the EMG sensors and balance sensing unit.

For example, movement and action instructions are provided to a patient in visual cues projected on a surface in patient's vicinity; while data regarding a patient's movements and actions are collected by said camera sensor 102, the surface EMG sensors 103, 104, and balance sensing unit. In some embodiments, audible cues are also utilized to provide instructions for the patient.

Among others, the computerized device 105 collects the following types of data: room area dimensions and characteristics (e.g., usable floor area, properties of objects such as chairs, tables, balls, surfaces colors and textures, lighting in the vicinity, etc.), patient's physical characteristics (e.g., for example, height, weight, limb length, and etc.), patient's movement in area, patient's movement and action duration, distance of patient relative to a certain point in area, EMG signals, balance status, patient's stride length, number of steps per exercise, length of practice and etc.

The computerized device 105 may be connected by wire or by wireless network protocols such as Bluetooth and WIFI. The computer that processes the data may be the computerized device 105 or a distant server connected to it that serves data from multiple users geographically distant from each other, at which case, the computerized device may serve as a client computer for collecting the data, transmitting it for processing to the server computer, and for receiving 235 and displaying the exercise/practice and performance data to user.

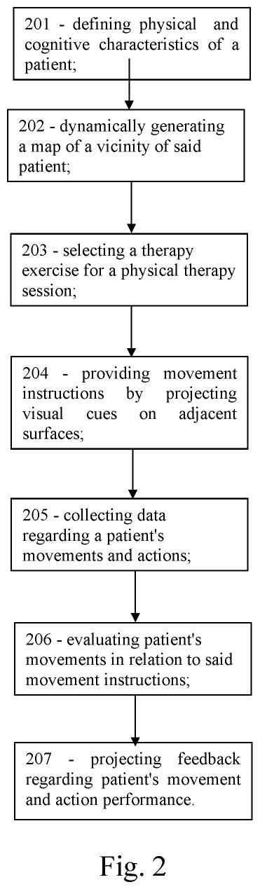

FIG. 2 depicts an exemplary method for physical therapy in augmented reality of an embodiment of the present invention.
a. The method may start with defining physical and cognitive characteristics of a patient based on data captured with at least one camera sensor (text box 201). The physical and cognitive characteristics may include for example, age, gender, height, weight, shoe size, length of limbs, reaction time, action data. The physical and cognitive characteristics may include diagnosis, prognosis, recent clinical events or visits to health care institutions including doctors visits, hospitalization period, cause, outcomes, body's temperature, pulse rate, blood pressure, respiration rate, BMI (body mass index). Some of the data can come from external databases such as for example, patient's medical file, or through measurement algorithms on data received from various sensors including the camera sensor, EMG, and the balance sensing unit.
b. The method may proceed with dynamically generating a map of a vicinity of said patient including floor area and object properties data by for example, generating a Point Cloud set up in real time using the camera sensor (text box 202).
c. The method may proceed with selecting a therapy exercise for a physical therapy session based on: the physical and cognitive characteristics of said patient, clinical information of said patient, the dynamic map of the vicinity of said patient, and dependency conditions of said therapy exercise that are selected from the group comprising: minimum floor area, maximum floor area, properties of objects in the area surfaces colors and textures in the area, lighting in the vicinity (text box 203). In some embodiments of the present invention, the selected therapy exercise may be adjusted to conform to the usable area adjacent to the patient.
d. The method may proceed with providing movement and action instructions by projecting visual cues on adjacent surfaces to a patient using said at least one projector (text box 204).
   The term ‘action’ in the context of the present disclosure refers to a physical task to be performed by a patient, e.g., for example, move to a position, pick an object.
e. The method may proceed with collecting data regarding a patient's movements and activity data from said at least one motion capture sensor and at least one surface EMG sensor balance sensing unit (text box 205).
f. The method may proceed with evaluating patient's movements and action data in relation to said movement and action instructions (text box 206);
g. The method may proceed with projecting feedback regarding patient's performance on adjacent surfaces (text box 207).

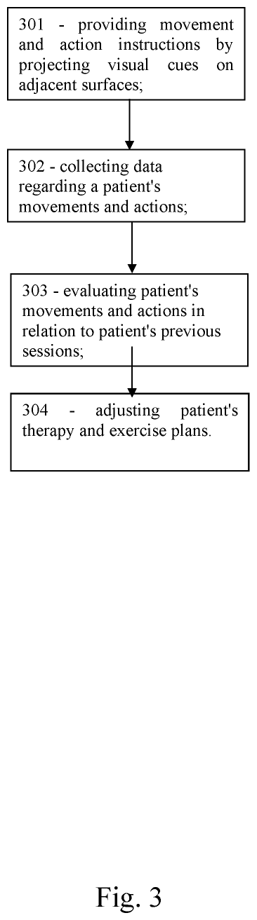

FIG. 3 depicts an exemplary method of the present invention.
a. The method may start with providing movement and action instructions by projecting visual cues on adjacent surfaces to a patient using said at least one projector (text box 301);
b. The method may proceed with collecting data regarding a patient's movements and action data from said at least one camera sensor, at least one surface EMG sensor, and balance sensing unit (text box 302);
c. The method may proceed with evaluating patient's movements and action data in relation to patient's previous sessions with the system (text box 303);
d. The method may proceed with adjusting patient's therapy and exercise plans (text box 304).

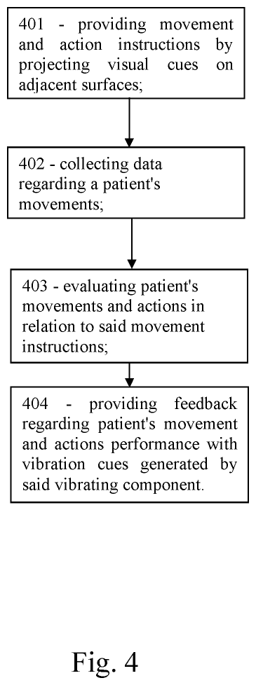

FIG. 4 depicts an exemplary method of the present invention.
a. The method may start with providing movement and action instructions by projecting visual cues on adjacent surfaces to a patient using said at least one projector (text box 401);
b. The method may proceed with collecting data regarding a patient's movements and action data from said at least one camera sensor and at least one surface EMG sensor, balance sensing unit (text box 402);
c. The method may proceed with evaluating patient's movements and action data in relation to said movement and action instructions (text box 403);
d. The method may proceed with providing feedback regarding patient's movement and action with vibration cues generated by said vibrating component (text box 404).

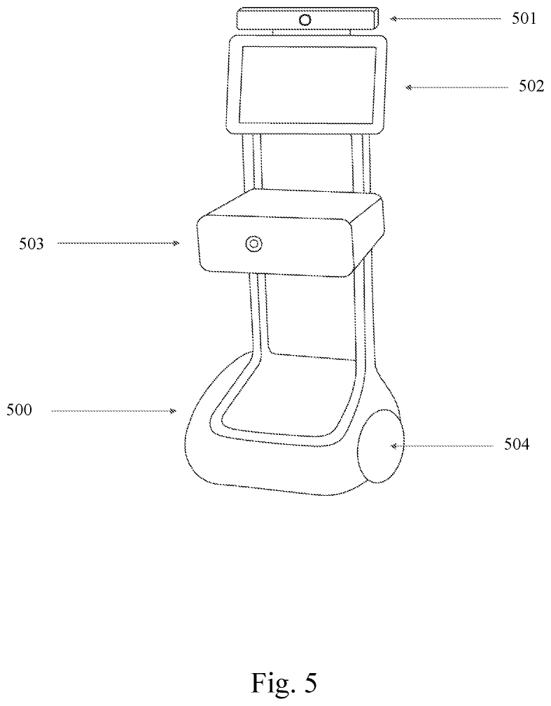

FIG. 5 depicts an exemplary configuration of a portable computerized device 500 consistent with the present invention. As can be seen the components of the device are integrated. Presented is an exemplary configuration comprising camera unit 501, and a projector 503.

This embodiment further comprises a screen 502 for presenting instructions and statistical data regarding the patient and wheels 504 for moving the device as required.

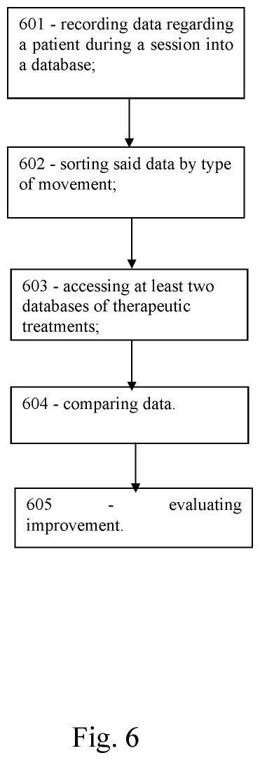

FIG. 6 depicts a method for evaluating patient's movements.
a. The method may start with recording data regarding a patient's movements and activity during a therapeutic session into a database (text box 601).
b. The method may proceed with sorting said data by type of movement and actions (text box 602).
   For example: data that was collected during an attempt to perform a step with left leg will be recorded in a single column of said database.
c. The method may proceed with accessing at least two databases with recordings of therapeutic treatments of said patient (text box 603).
   For example: accessing the database of the present treatment and the 4 last treatments.
d. The method may proceed with comparing data recorded in accessed databases in chosen type of movement or actions (text box 604).
e. The method may proceed with evaluating improvement in said chosen type of movement or actions in comparison to previous recording of a therapeutic session (text box 605).

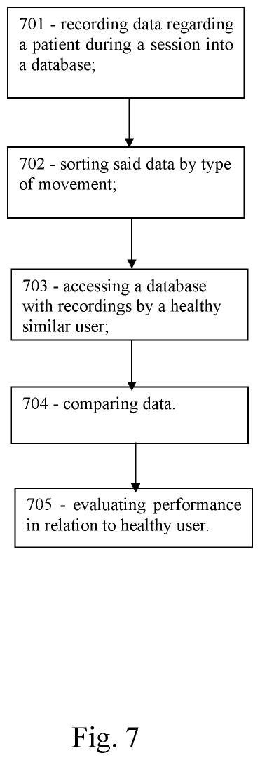

FIG. 7 depicts a method for evaluating patient's movements and actions.
a. The method may start with recording data regarding a patient's movements and actions during a therapeutic session into a database (text box 701).
b. The method may proceed with sorting said data by type of movement and actions (text box 702).
c. The method may proceed with accessing a database with recordings of type of movements and actions by a healthy patient having similar characteristics (e.g. height, weight, age, gender) to said patient (text box 703).
d. The method may proceed with comparing data recorded in accessed databases in chosen type of movement and actions (text box 704).
e. The method may proceed with evaluating performance of said patient in relation to healthy patient performing said chosen type of movement and actions (text box 705).

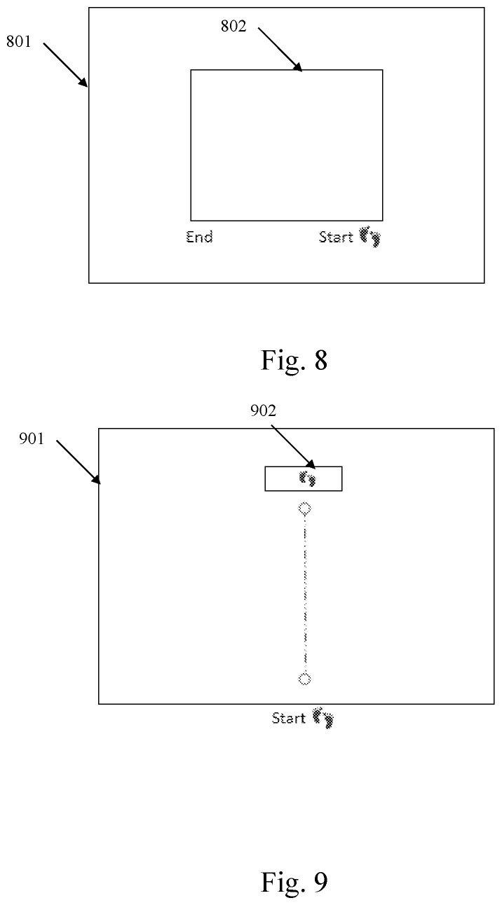

FIG. 8 depicts a projection of visual cues on an interaction area 801. The projector (not shown) projects an image of a square 802 on top of the interaction area 801. The patient is instructed through various audio-visual cues to walk along the lines of the square.

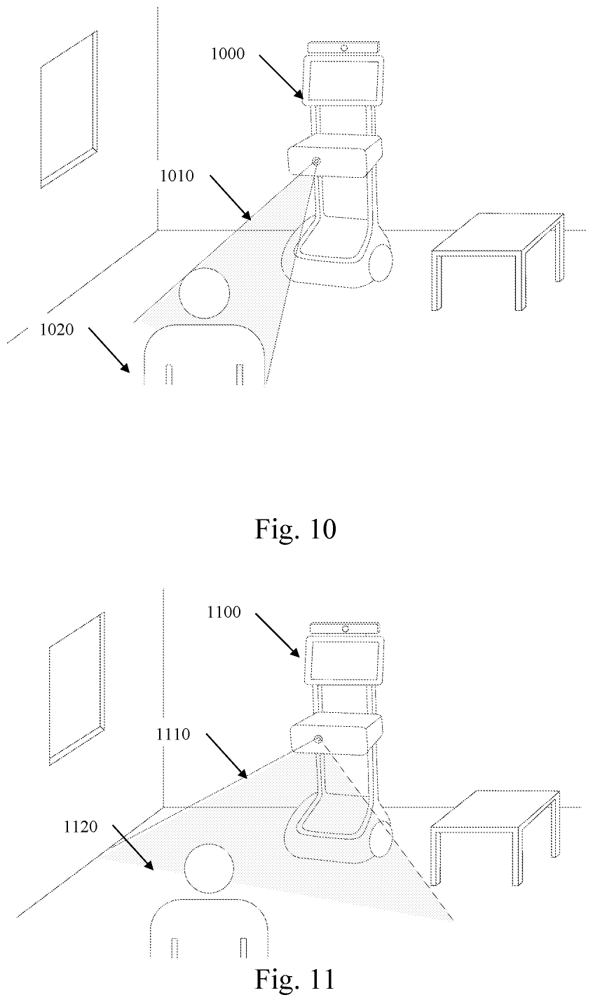

FIG. 9 depicts a projection of visual cues on an interaction area 901. Within the interaction area there is a stair 902. The projector (not shown) projects an image on top of the stair 902. The patient is instructed through various audio-visual cues to walk to the stair and step upon it. FIG. 10 depicts an exemplary computerized device of the present invention. The device 1000 is scanning 1010 the patient 1020 for defining his physical characteristics.

FIG. 11 depicts an exemplary computerized device of the present invention. The device 1100 is scanning 1110 the patient's vicinity for dynamically generating a map of the room and usable area for exercises 1120.

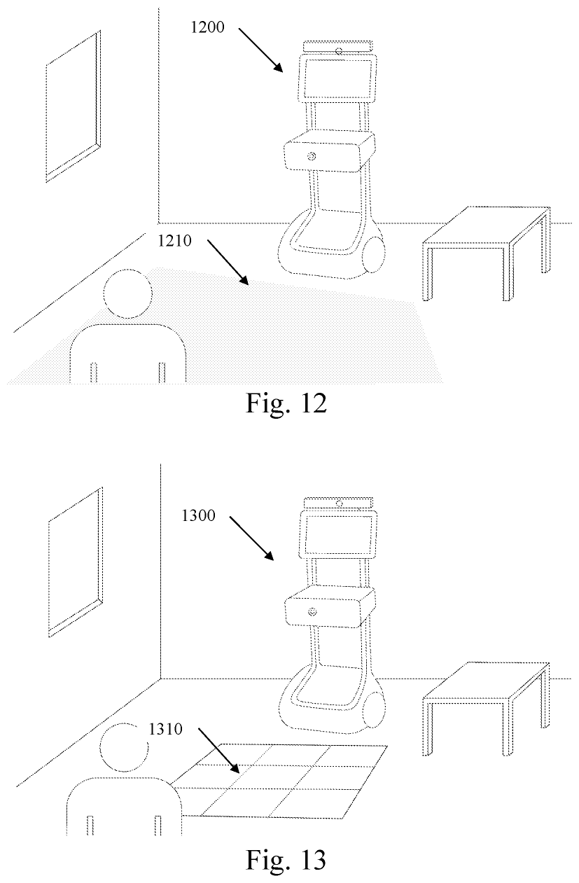

FIG. 12 depicts an exemplary computerized device of the present invention. The device 1200 has dynamically generated a map of the room and usable area for exercises 1210.

FIG. 13 depicts an exemplary computerized device of the present invention. The device 1300 projects movement and action instructions 1310 on the usable area in the patient's vicinity.

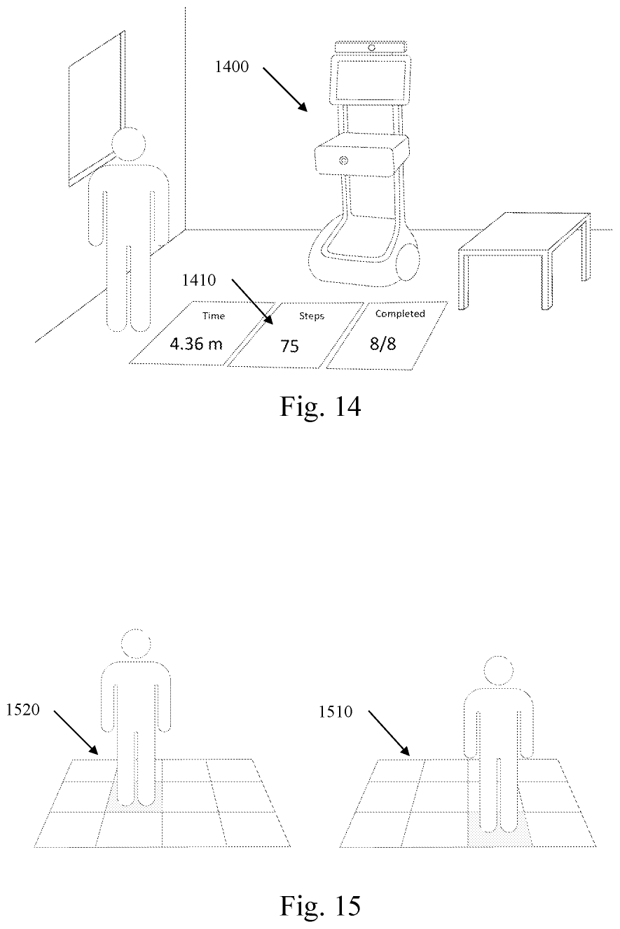

FIG. 14 depicts an exemplary computerized device of the present invention. The device 1400 projects exercise results and statistics 1410 on the usable area in the patient's vicinity.

FIG. 15 depicts an exemplary exercise to be performed with an exemplary computerized device of the present invention. The exercise includes several squares projected on the usable area in the patient's vicinity. The computerized device identifies when the patient steps on one of the squares and highlights 1510 1520 it in a different color. The computerized device may instruct the patient to move to another square by highlighting it in yet another color.

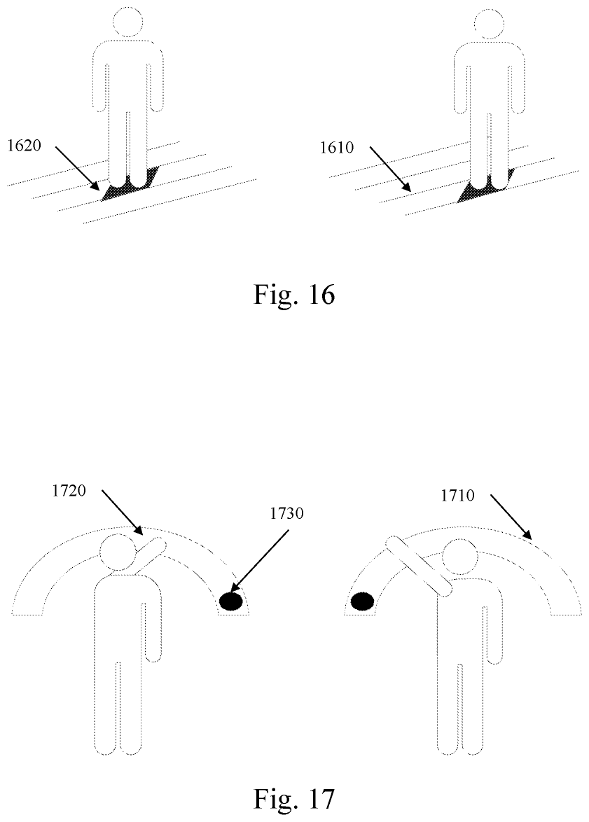

FIG. 16 depicts an exemplary exercise to be performed with an exemplary computerized device of the present invention. The exercise includes several parallel lines projected on the usable area in the patient's vicinity. The computerized device identifies when the patient crosses between areas delimited by the lines and highlights 1610 1620 the area in a different color. The computerized device may instruct the patient to move to another area by highlighting it in yet another color.

FIG. 17 depicts an exemplary exercise to be performed with an exemplary computerized device of the present invention. The exercise includes a gauge-like shape 1710 projected on the usable area in the patient's vicinity. The computerized device identifies the position of patient's arm 1720 in relation to the shape and instructs him to move the hand by moving an indicator 1730 in the gauge.

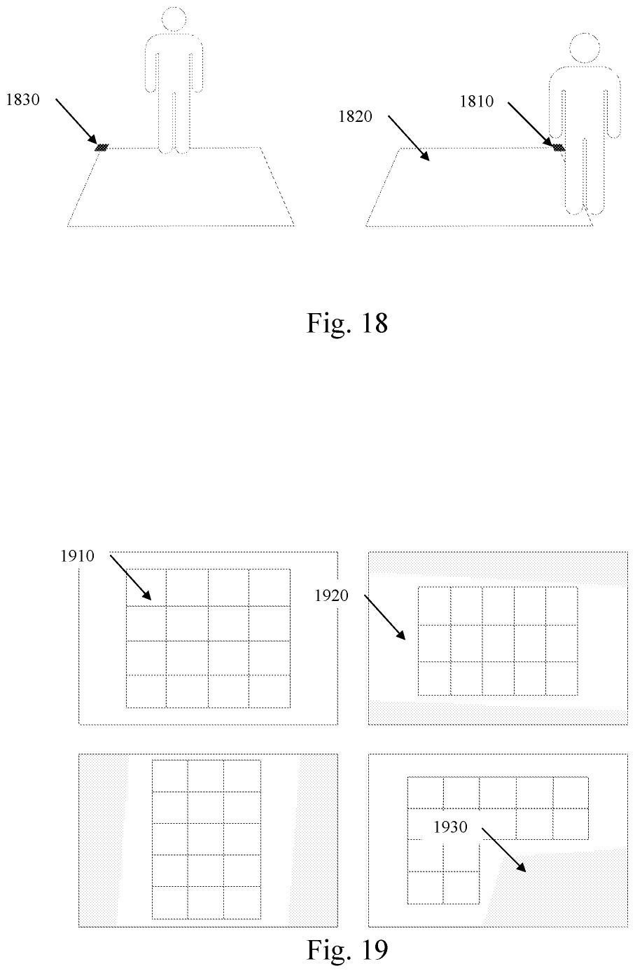

FIG. 18 depicts an exemplary exercise to be performed with an exemplary computerized device of the present invention. The exercise includes a square 1810 projected on the usable area 1820 in the patient's vicinity. The computerized device identifies when the patient steps on the squares and changes its position to another location 1830 where the patient is instructed to move.

FIG. 19 depicts exemplary exercises to be performed with an exemplary computerized device of the present invention. The computerized device may select and adjust an exercise based on the dynamic map of the vicinity of said patient. As can be seen, the squares 1910 included in the exercise are arranged so to fit to the usable area 1920 that is represented by white area, whereas unusable area 1930 represented by grey. Unusable area may be for example an area blocked by obstacles, or an uneven floor if the exercise requires that the floor to be horizontal.

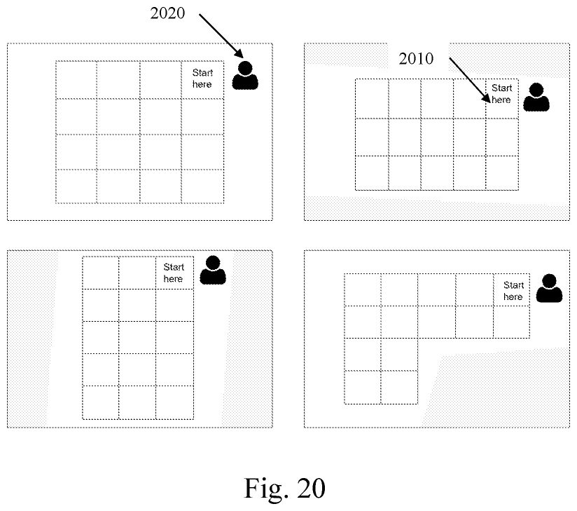

FIG. 20 depicts exemplary exercises to be performed with an exemplary computerized device of the present invention. The computerized device may select and adjust an exercise based on the dynamic map of the vicinity of said patient and patient's position 2010 relative to the exercise area. The computerized device may adjust the instructions and the exercise based on the position of the patient, for example, projecting an indicator 2020 in the square closest to the patient.

The foregoing description and illustrations of the embodiments of the invention has been presented for the purposes of illustration. It is not intended to be exhaustive or to limit the invention to the above description in any form. Any term that has been defined above and used in the claims, should be interpreted according to this definition.

1. A computerized device for physical therapy in augmented reality, comprising:
at least one camera sensor and at least one projector for projecting visual cues on surfaces in a patient's vicinity;
wherein, processing circuitry of said computerized is configured to:
define physical characteristics of said patient based on data captured with said at least one camera sensor;
dynamically generate a map of said vicinity of said patient including floor area and object properties data;
select and adjust a therapy exercise for a physical therapy session based on: the physical characteristics of said patient, clinical information of said patient, the dynamic map of the vicinity of said patient, and dependency conditions of said therapy exercise that are selected from the group comprising: minimum floor area, maximum floor area, properties of objects in the area, surface colors and textures in the area, and lighting in the vicinity;
instruct said at least one projector to project visual cues of the selected therapy exercise as movement and action instructions on said surfaces in said patient's vicinity.

2. The computerized device of claim 1, comprising instructions that cause the processing circuitry of said computerized device to:
collect data regarding said patient's movements and action data in relation to said visual cues from said at least one camera sensor, an at least one surface EMG sensor, and a balance sensing unit, and store the collected data in a database;
compare said collected data with data of movements and activity data collected during previous sessions of said patient;
instruct said at least one projector to project visual cues on said surfaces in said patient's vicinity as feedback regarding said patient's performance in relation to said movement and action instruction; and
based on comparison of said collected data to previously collected data, adjust said patient's therapy plan, movement and action instructions.

3. The computerized device of claim 1, wherein said data is processed and analyzed into a representation of said patient's status and progress, further providing suitable therapy and exercise plans.

4. A method for physical therapy in augmented reality, comprising steps of:
defining physical characteristics of a patient based on data captured with at least one camera sensor;
dynamically generating a map of a vicinity of said patient including floor area and object properties data;
selecting and adjusting a therapy exercise for a physical therapy session based on: the physical characteristics of said patient, clinical information of said patient, the dynamic map of the vicinity of said patient, and dependency conditions of said therapy exercise that are selected from the group comprising: minimum floor area, maximum floor area, properties of objects in the area, surfaces colors and textures in the area, and lighting in the vicinity;
providing movement and action instructions according to the selected therapy exercise by projecting visual cues on adjacent surfaces to said patient using at least one projector;
collecting data regarding the patient's movements and action from at least one camera sensor, at least one balance sensing unit, and at least one surface EMG sensor;
evaluating said patient's movements and actions in relation to said movement and action instructions;
providing feedback regarding said patient's movement and actions by projecting feedback on said adjacent surfaces; and
adjusting said patient's therapy and exercise plans.

5. The method of claim 4, wherein evaluating said patient's movement and actions comprises:
recording data regarding said patient's movements and actions during said therapy session into a database in said computer;
sorting said data by type of movement and action;
accessing at least two databases with recordings of previous therapy sessions of said patient;
comparing data recorded in the accessed databases in chosen type of movement or action;
evaluating improvement in the chosen type of movement and action in comparison to a previous recording of said therapy session.

6. The method of claim 5, comprising:
evaluating performance of said patient in relation to a healthy patient performing the chosen type of movement and action.
